(19) United States
(12) Patent Application Publication
(10) Pub. No.: US 2022/0410113 A1
(43) Pub. Date: Dec. 29, 2022

(54) SYNTHETIC DATA STORAGE SYSTEM BASED ON ATTRIBUTES OF ARECACEAE

(71) Applicant: Seagate Technology LLC, Fremont, CA (US)

(72) Inventor: Padmaja Kannan, Singapore (SG)

(21) Appl. No.: 17/844,278

(22) Filed: Jun. 20, 2022

Related U.S. Application Data
(60) Provisional application No. 63/213,946, filed on Jun. 23, 2021.

(51) Int. Cl. B01J 19/00; G06N 3/12; C09K 5/20; G16B 35/00

(52) U.S. Cl. CPC B01J 19/0046; G06N 3/123; C09K 5/20; G16B 35/00

(57) ABSTRACT

A data storage medium includes a substrate. The data storage medium also includes an antifreeze layer coated on at least one surface of the substrate. The data storage medium further includes multiple storage containers located on the substrate. The multiple storage containers store different combinations of plant-based molecules representing data.

### CROSS-REFERENCE TO RELATED APPLICATION

This application claims priority to U.S. Provisional Application No. 63/213,946, filed on Jun. 23, 2021, the content of which is hereby incorporated in its entirety.

### SUMMARY

In one embodiment, a data storage system is provided. The data storage system includes a freeze-tolerant data storage medium. The freeze-tolerant data storage medium has a grid with a plurality of grid elements. Each grid element of the plurality of grid elements includes a storage container. The data storage system also includes a molecule generator that is configured to generate a plurality of different plant-based molecules representing data. The data storage system further includes a molecule depositor, which is coupled to the molecule generator. The molecule depositor is configured to deposit different combinations of the plurality of different plant-based molecules into different storage containers of the plurality of storage containers.

In another embodiment, a method is provided. The method includes providing a substrate, and coating at least one surface of the substrate with an antifreeze layer. The method also includes providing a plurality of storage containers on the substrate. The plurality of storage containers is configured to store different combinations of a plurality of different plant-based molecules representing data.

In yet another embodiment, a data storage medium is provided. The data storage medium includes a substrate, and an antifreeze layer coated on at least one surface of the substrate. The data storage medium also includes a plurality of storage containers on the substrate. The plurality of storage containers is configured to store different combinations of a plurality of different plant-based molecules representing data.

Other features and benefits that characterize embodiments of the disclosure will be apparent upon reading the following detailed description and review of the associated drawings.

### BRIEF DESCRIPTION OF THE DRAWINGS

FIG. 1 is a diagrammatic illustration of a synthetic data storage system in accordance with one embodiment.

FIG. 2 is a diagrammatic illustration showing molecules generated from amino acids.

FIG. 3 is a diagrammatic illustration showing multiple categories of multi-component reactions (MCRs).

FIG. 4 is a diagrammatic illustration of MCR process equipment in accordance with one embodiment.

FIG. 5 shows an example in which amino acids are combined to form a unique molecule.

FIG. 6 shows an example in which amino acids are combined to form unique molecules that together represent a word.

FIG. 7 is a diagrammatic illustration showing an example of generation and storage of data in accordance with one embodiment.

FIG. 8 is a diagrammatic illustration of a spectrometer for identifying stored molecules in accordance with one embodiment.

### DETAILED DESCRIPTION OF ILLUSTRATIVE EMBODIMENTS

Embodiments of the disclosure provide synthetic data storage systems that are based in part on physical and chemical properties of Arecaceae (a family of perennial flowering plants).

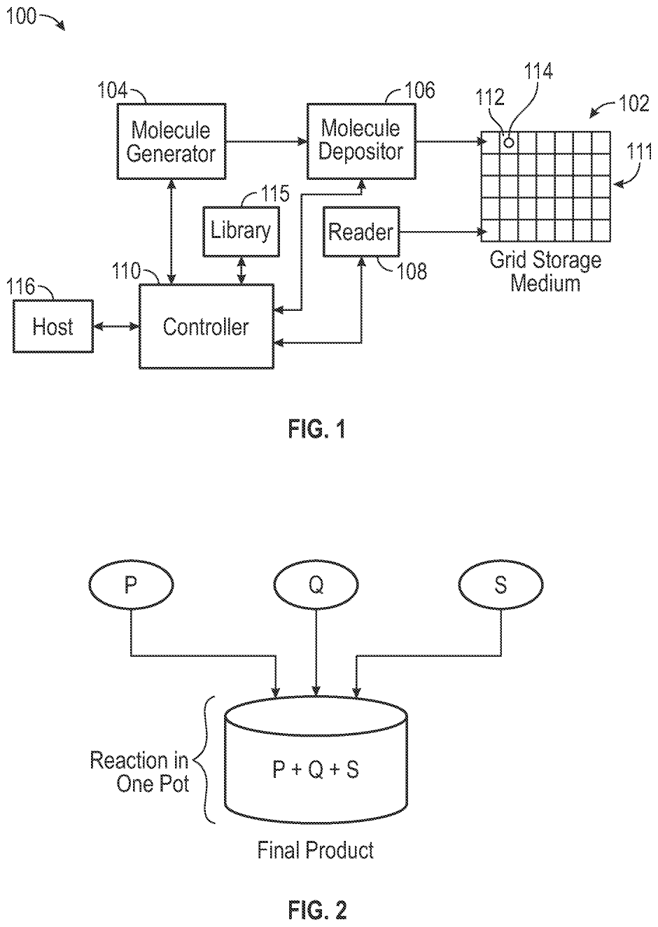

In some embodiments, molecules that are similar to molecules found in Arecaceae are generated and utilized to represent data, and media having antifreeze properties of Arecaceae are developed and utilized to store the generated molecules that represent the data. Prior to providing additional detail regarding different embodiments of the disclosure, an example general embodiment is described below in connection with FIG. 1.

It should be noted that like reference numerals are used in different figures for same or similar elements. It should also be understood that the terminology used herein is for the purpose of describing embodiments, and the terminology is not intended to be limiting. Unless indicated otherwise, ordinal numbers (e.g., first, second, third, etc.) are used to distinguish or identify different elements or steps in a group of elements or steps, and do not supply a serial or numerical limitation on the elements or steps of the embodiments thereof. For example, “first,” “second,” and “third” elements or steps need not necessarily appear in that order, and the embodiments thereof need not necessarily be limited to three elements or steps. It should also be understood that, unless indicated otherwise, any labels such as “left,” “right,” “front,” “back,” “top,” “bottom,” “forward,” “reverse,” “clockwise,” “counter clockwise,” “up,” “down,” or other similar terms such as “upper,” “lower,” “aft,” “fore,” “vertical,” “horizontal,” “proximal,” “distal,” “intermediate” and the like are used for convenience and are not intended to imply, for example, any particular fixed location, orientation, or direction. Instead, such labels are used to reflect, for example, relative location, orientation, or directions. It should also be understood that the singular forms of “a,” “an,” and “the” include plural references unless the context clearly dictates otherwise.

It will be understood that, when an element is referred to as being “connected,” “coupled,” or “attached” to another element, it can be directly connected, coupled or attached to the other element, or it can be indirectly connected, coupled, or attached to the other element where intervening or intermediate elements may be present. In contrast, if an element is referred to as being “directly connected,” “directly coupled” or “directly attached” to another element, there are no intervening elements present. Drawings illustrating direct connections, couplings or attachments between elements also include embodiments, in which the elements are indirectly connected, coupled or attached to each other.

FIG. 1 is a diagrammatic illustration of a synthetic data storage system 100 in accordance with one embodiment. Synthetic data storage system 100 includes one or more data storage media 102, a molecule generator 104, a molecule depositor 106, a reader 108 and a controller 110 that manages data storage and retrieval operation in synthetic data storage system 100. In the embodiment of FIG. 1, synthetic data storage system 100 is shown coupled to a host 116 from which controller 110 can receive read and/or write commands. Host 116 may be coupled to controller 110 by a wired connection, or may be wirelessly coupled to controller 110. Host 116 represents any type of computing system that is configured to send data to and receive data from a data storage device or system. Examples of host 116 include cloud computing environments, servers, desktop computers, laptop computers, mobile phones, tablet computers, televisions, automobiles, surveillance cameras, or any other type of mobile or non-mobile computing device that is configured to read and write data. In some embodiments, a host such as 116 may not be involved, and controller 110 may directly transfer data from/to a memory or tangible medium that stores binary data to/from the data storage medium 102, which, as will be described below, stores information as molecules. In such embodiments, controller 110 may be equipped with circuitry configured to read data from and/or write data to the memory or tangible medium.

In data storage system 100, molecule generator 104 and molecule depositor 106 together act as a writer to store information as molecule combinations on data storage medium 102. Reader 108 identifies the molecule combinations on the data storage medium 102, and thereby retrieves the stored information from the data storage medium 102. Controller 110 may include one or more processors and one or more memories that store program code having instructions that are executable by the processor(s). Controller 110, which is coupled to molecule generator 104, molecule deposition 106 and reader 108, may control operations of those components.

In embodiments of the disclosure, the molecules generated by molecule generator 104 and deposited on data storage medium 102 by molecule depositor 106 are molecules that are formed using amino acids found in Arecaceae, and the data storage medium 102 has antifreeze or freeze-tolerant properties of (or similar to) Arecaceae. Details regarding antifreeze properties of Arecaceae are provided further below.

In data storage system 100, freeze-tolerant data storage medium 102 has a grid 111 including multiple grid elements 112. In some embodiments, each of the grid elements 112 includes a storage container 114 that is configured to store molecules deposited by depositor 106. In one embodiment, each storage container 114 may include an adhesive that holds the molecules in place when deposited into the container 114. In other embodiments, no containers 114 are used, and the molecules may be directly deposited on the grid elements 112. In such embodiments, the adhesive that is configured to hold the molecules in place when deposited may be applied directly on the grid elements 112. Details regarding how the molecules are generated are provided further below.

Synthetic data storage system 100 may include a library of characters 115 that maps characteristics of different molecules and/or molecule combinations to different combinations of bits. Thus, when binary data arrives to the synthetic data storage system 100 from a host 116 for storage, molecule types corresponding to the binary data are first identified from the library 115 by controller 110. Then, based on the molecule types identified by controller 110, molecule generator 104 generates the molecules by one or more chemical reactions and provides them to molecule depositor 106. Molecule depositor 106 deposits the molecules in storage containers 114. To carry out a read operation for the stored data (for example, in response to a read command received from host 116), controller 110 directs reader 108 to the container(s) 114 that include molecules that represent the requested information. The reader 108 identifies the molecules, and returns the identified information to the controller 110. The controller 110 converts the identified information into binary data, and returns the data to the host 116.

In the embodiment shown in FIG. 1, the writing elements (molecule generator 104, molecule depositor 106 and write control functionality provided by controller 110) and reading elements (reader 108 and read control functions provided by controller 110) are shown within a single synthetic data storage system 100. However, in some embodiments, write and read operations may be carried out at separate locations, and separate controllers may be employed to provide the read and write functionalities. Thus, data storage medium 102 may be attached or coupled to the writing elements for write operations to be carried out, and then detached or decoupled from the write elements. The detached data storage medium 102 with the deposited molecules may be stored for a long period of time. In some embodiments, to preserve the molecules for a long period of time, the data storage medium 102 with the deposited molecules may be immersed in a cooling liquid. When the stored information is to be read from the data storage medium 102, the data storage medium may be removed from the liquid, and then attached, coupled, or positioned such that read elements are able to identify the molecules on the data storage medium 102, thereby enabling information recovery.

In one embodiment, the data storage medium 102 includes a glass plate, with one or more surfaces of the glass plate coated with an antifreeze layer (e.g., an antifreeze protein (AFP)) similar to chemical properties of Arecaceae. Physical and chemical properties of Arecaceae are listed below in tables 1 and 2, respectively.

TABLE 1

| | Physical properties | | | |
|---|---|---|---|---|
| | | | Tensile | Young's |
| | Density | Elongation | strength | modulus |
| Fiber | (g/cm3) | (%) | (MPa) | (GPa) |
| Palm leaf stalk | 1-1.2 | 2-4.50 | 97-196 | 2.50-5.40 |
| Palm leaf sheath | 1.20-1.30 | 2.84 | 220 | 4.8 |
| Palm petiole | 0.7-1.55 | 25 | 248 | 3.24 |
| Palm fruit | 1.09 | 28 | 423 | 6.-8. |
| Coir | 1.15-1.2 | 30 | 175 | 4.-6. |
| Pineapple leaf | 0.80-1.60 | 14.5 | 144 | 400-627 |

Young's modulus is a mechanical property that measures stiffness of a solid material. It defines a relationship between stress (force per unit area) and strain (proportional deformation) in a material in a linear elasticity regime of a uniaxial deformation. The international system (SI) unit of Young's modulus is pascal (GPa in Table 1 is gigapascal).
Tensile strength is a resistance of a material to breaking under tension. In other words, the tensile strength of a material is the maximum amount of tensile stress that it can take before failure, for example breaking. The SI unit of Young's modulus is pascal (MPa in Table 1 is megapascal).
Elongation is a state, act, or process of lengthening.
Density of a substance is its mass per unit volume (grams(s)/cubic centimeter (cm³) in Table 1).

TABLE 2

| | Chemical properties | | | |
|---|---|---|---|---|
| | Cellulose | Hemi cellulose | Lignin | Wax |
| Fiber | (%) | (%) | (%) | (%) |
| Palm leaf stalk | 40-52 | 42-43 | — | — |
| Palm leaf sheath | 28 | 25 | 45 | — |
| Palm petiole | 30 | 14 | 28 | — |
| Palm fruit | 53 | 12 | 21 | 0.8 |
| Coir | 32-43 | 0.15-0.25 | 40-45 | — |
| Pineapple leaf | 70-83 | — | 5-12.7 | — |

Cellulose content is responsible for long fiber chains that range from 28%-53% for palm fibers.
Hemi-cellulose leads to disintegration of cellulose microfibrils that decrease the fiber strength. For palm, hemi-cellulose ranges from 12%-43%. Microfibrils is used as a general term to describe the structure of protein fiber, e.g., hair.
Lignins help in the formation of cell walls, especially in wood and bark, because they lend rigidity and do not rot easily.
Plants secrete waxes into and on the surface of their cuticles as a way to control evaporation, wettability and hydration.

Plants such as Arecaceae have their own deoxyribonucleic acid (DNA) and their properties such as density and durability are some of the premises that were considered for the disclosure. Regarding density, research papers indicate that one gram of DNA can potentially store 215 petabytes of data. An average hard disk drive in a laptop can house just one millionth of that amount. Thus, as indicated above, embodiments of the disclosure encode data at a molecule level (using synthesized plant-based molecules) and store the molecules representing the large amounts of data in a medium that extends the data shelf life. Regarding durability, the natural physical and chemical properties of palm leaves, if preserved under the right conditions, can increase the shelf life of the medium for 600 years or more.

Palm leaves includes AFPs, also known as thermal hysteresis proteins, which are compound proteins (e.g., a protein complex combining amino acids with other substances, usually sugar), which have the following properties:
A high concentration of amino acid threonine.
A naturally formed AFP is not hazardous and toxic like chemical antifreeze used in cars (ethylene glycol).
A high rich cellulose content and a relatively low lignin content in palm fibers ensure substantial mechanical strength.
Tensile values of palm fibers are significantly higher than any other natural fibers and can be used for reinforcement in a polymer matrix.
Palm fruit fiber reinforced polyester composite has an appreciable tensile strength (e.g., 100-200 MPa). The tensile strength is based on the size of a medium (e.g., 50*50 by 2 mm thickness or 20*60 by 2 mm thickness). In Table 1 above (physical properties table), tensile strength varies for different fibers (e.g., variations in tensile strength from palm leaf stalk to coir).

Antifreeze prevents growth of crystals when a data storage medium (e.g., 102 of FIG. 1) is immersed in any cooling liquid. Accordingly, as indicated above, one or more surfaces of the data storage medium are designed such that they are freeze tolerant and freeze avoidant. Thus, as noted above in the description of FIG. 1, in one embodiment, at least one surface of the data storage medium 102 is coated with an AFP. Further, as noted above, data storage medium 102 has a grid 111 that includes grid elements 112, which are each configured to store (or include containers 114 that are configured to store) synthesized plant-based molecules. Details regarding formation of the plant-based molecules from amino acids are provided below.

Unlike polymers of nucleotides where alphabets can be formed from 4 nucleotide bases A, T, G and C, polymers of amino acids (known as proteins) have 22 flavors. Each synthesized small plant-based molecule is created using amino acids. Amino acids utilized to generate the plant-based molecules in some embodiments are listed below in table 3.

TABLE 3

| | | | Molecular |
|---|---|---|---|
| | Three | One | weight in |
| Amino Acid Name | Letter Code | Letter Code | Dalton (Da) |
| alanine | ala | A | 89 |
| arginine | arg | R | 174 |
| asparagine | asn | N | 132 |
| aspartic acid | asp | D | 133 |
| cysteine | cys | C | 121 |
| glutamine | gln | Q | 146 |
| glutamic acid | glu | E | 147 |
| glycine | gly | G | 147 |
| histidine | his | H | 155 |
| isoleucine | ile | I | 131 |
| leucine | leu | L | 131 |
| lysine | lys | K | 146 |
| methionine | met | M | 149 |
| phenylalanine | phe | F | 165 |
| proline | pro | P | 115 |
| serine | ser | S | 105 |
| threonine | thr | T | 119 |
| tryptophan | trp | W | 204 |
| tyrosine | tyr | Y | 181 |
| valine | val | V | 117 |

Dalton (Da) is an alternate name for an atomic mass unit, and a kilodalton (kDa) is 1,000 daltons. Thus, a protein with a mass of 64 kDa has a molecular weight of 64,000 grams per mole.

Each amino acid has a spectral color, and when a molecule is created with amino acid combinations, that molecule is associated with a color. In some embodiments, each molecule is created using a multi-component reaction (MCR) process, and each molecule represents an alphabet. Accordingly, a word library may be created from molecules.

Some reasons for choosing an MCR process for generating molecules are as follows:

1) MCRs are convergent reactions in which three or more components react to form a product.
2) MCRs involve a single operational step, and may be carried out in a single pot/container. FIG. 2 is a diagrammatic illustration showing amino acids P, Q and S introduced into a single container/pot in which an MCR takes place to produce a final product including multiple molecules, with each molecule including an amino acid combination P+Q+S.
3) An MCR reduces waste generation due to its convergent nature and its single operational step.
4) An MCR does not generate a by-product, and is considered to be an eco-friendly reaction system.
5) MCRs are substantially atomically balanced (e.g., a number of atoms of any given element does not change in any reaction, which maximizes the incorporation of all materials used in the process).
6) Due to weak negative reactions, MCRs are usually safe processes.
7) MCRs save time, and are energy efficient.

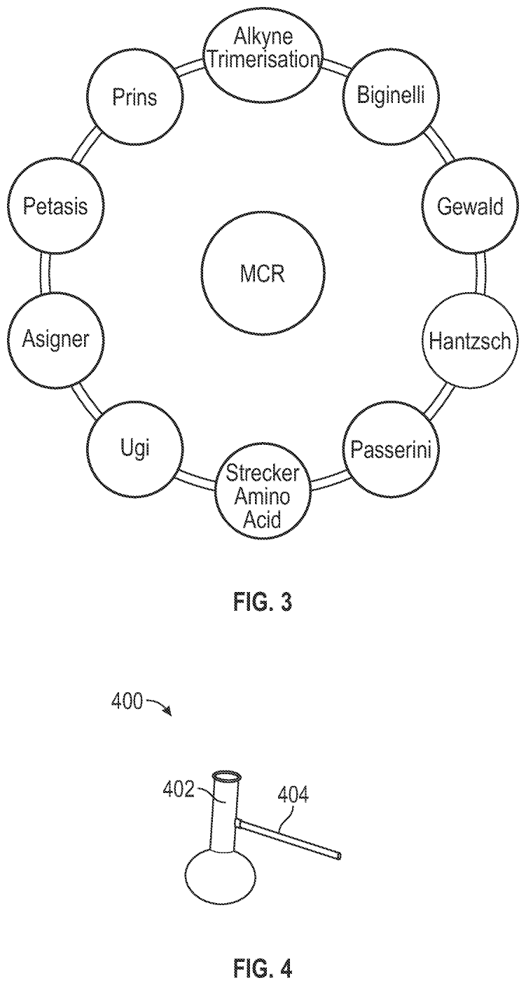

FIG. 3 is a diagrammatic illustration showing multiple categories/reactions of MCRs. A suitable reaction combination may be obtained by combining any categories/reactions of MCRs in FIG. 3. Selecting a suitable combination of categories/reactions of MCRs from the categories/reactions listed in FIG. 3 may depend on any individual category's/reaction's properties, and whether infusing a particular category/reaction of MCR with other categories/reactions will improve yield, efficiency, etc. For instance,
Hantzsch MCR has a low yield time and a high reaction time.
Strecker MCR produced better yield and consistency after repeated tests.
Ugi MCR gives the highest yields and a high atom balance. Ugi reactions are often used in the pharmaceutical industry to relatively rapidly produce large number of different compounds.
Passerini MCR may be carried out at a relatively low temperature.
Dumas may be utilized for determining a mass of a molecule.
   Accordingly, as indicated above, to improve yield and efficiency, chemical properties of different MCR reactions/categories are taken into consideration before building a final product.

FIG. 4 is a diagrammatic illustration of MCR process equipment 400 in accordance with one embodiment. MCR process equipment 400 may be included in, or serve as, molecule generator 104 of FIG. 1 in some embodiments. MCR process equipment 400 may include a laboratory flask 402 with a side tube 404. Amino acids are added to the flask 402, and MCR reactions are carried out. The resultant molecules from the chemical reaction are carried through the side tube 404 to a molecule depositor (such as 106 of FIG. 1), which deposits the molecules at predetermined locations on a data storage medium (such as 102 of FIG. 1). In some embodiments, the molecule depositor may include a spout (not shown) at the end of side tube 404. It should be noted that, in some embodiments of the disclosure, more complex equipment may be used to generate, siphon off, and introduce individual molecules into a depositor for deposition on a data storage medium (such as 102 of FIG. 1). For example, when the MCR process is carried out, individual molecules may be separated out using electrophoresis and/or chromatography. These are known processes, which may be automated. In general, electrophoresis involves the movement of charged molecules through a suspension (e.g., a gel) under the action of an electromotive force applied to electrodes in contact with the suspension. Pores in the suspension (e.g., gel) operate like a sieve, allowing smaller molecules to move faster than larger molecules. Chromatography generally involves separating molecules of interest from, for example, a liquid (mixture). This method has many techniques, one of which is ion-exchange where molecules are separated based on their surface charge values. As indicated above, these processes are known, and therefore additional details are not provided in the interest of brevity.

MCR methodology is considered as a sorted tool to create combinational libraries. Since MCR can generate diverse libraries, it can be considered as a tool to formulate unique molecules. As indicated above, in embodiments of the disclosure, MCR reactions are used to combine amino acids to make unique molecules. Each unique molecule represents a unique character and a group of molecules forms a word. Every different generated character can be associated with a unique color. For example, 26 alphabets=26 unique molecules, and each unique molecule is associated with a unique color. Thus, a library of words may be created based on the generated molecules. Examples for creating data from amino acids are provided below.

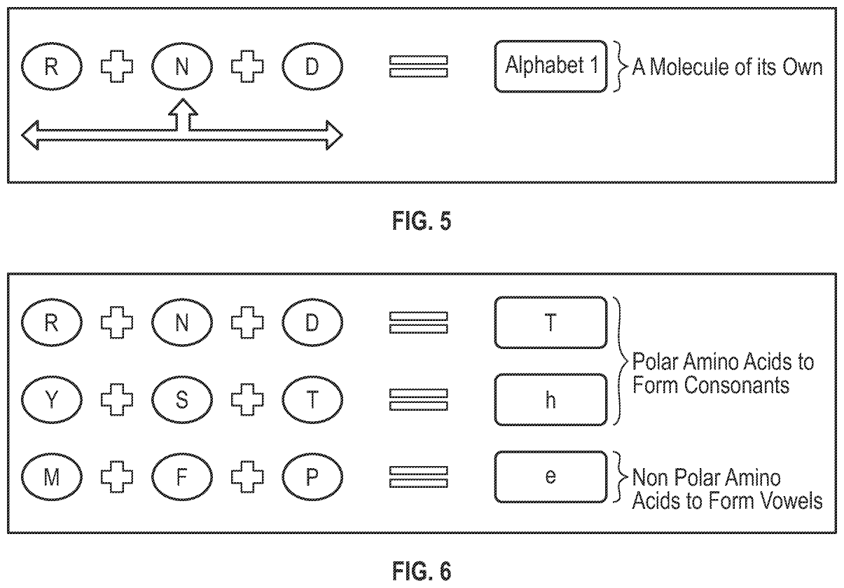

An amino acid has both a basic amine group and an acidic carboxylic acid group. The 20 amino acids in Table 3 are structured as polar and non-polar amino acids. Distinct letters may be formed from polar or non-polar amino acids. FIG. 5 shows an example in which amino acids R, N and D are combined to form a unique molecule, which represents a unique alphabet. In general, a character can be formed by combining any polar/non-polar amino acid. Vowels may be represented by certain combinations of molecules generated from the amino acids listed in Table 3. Data may be stored in single/three-digit codes instead of binary (1s and 0s). An example of how to write a word as a combination of molecules is provided below.

Consider the word “The,” which has the following binary equivalents of ASCII from an ASCII-binary character table:

### T is 01010100

h is 01101000
e is 01100101
As can be seen in FIG. 6, R, N and D, which are polar amino acids, are combined to form a unique molecule, which represents the alphabet “T.” Similarly, Y, S and T, which are also polar amino acids, are combined to form the alphabet “h.” Non-polar amino acids M, F and P are combined to form the alphabet “e.” In some embodiments, polar amino acids may be combined to form consonants, and non-polar amino acids may be combined to form vowels. A method for creating a molecule may be thought of converting amino acids into a binary format, and such a method may be automated.
To form a molecule (T) from R+N+D, 2 R, 2N and 1D with an additional standard component are utilized. A simple demonstration is as follows:

Convert R, N, D into binary equivalents

R is 01010010

N is 01001110

D is 01000100

As noted above, T is 01010100

R*R=01101001000100

(R*R)/N=01010110

01010110+N=010101100

010101100−D=01101000

01101000−00010100=01010100 (which is T)

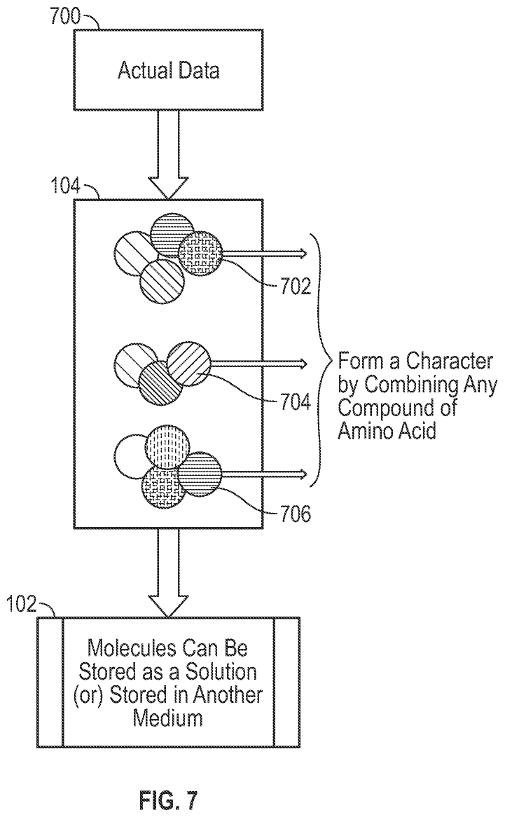

FIG. 7 is a diagrammatic illustration showing actual data (e.g., binary data for alphabets T, H and E) 700 provided to a molecule generator 104 where different molecules or combinations of molecules 702, 704 and 706 are generated from amino acids to represent alphabets T, H and E, respectively. Those molecules 702, 704 and 706 are stored on a data storage medium such as 102 in a manner described above in connection with FIG. 1. In addition to alphabets, amino acids may be combined to form groups of alphabets, characters, and words. For example, any word, such as the word “edge”, can be formed permanently using a particular amino acid compound mixture.

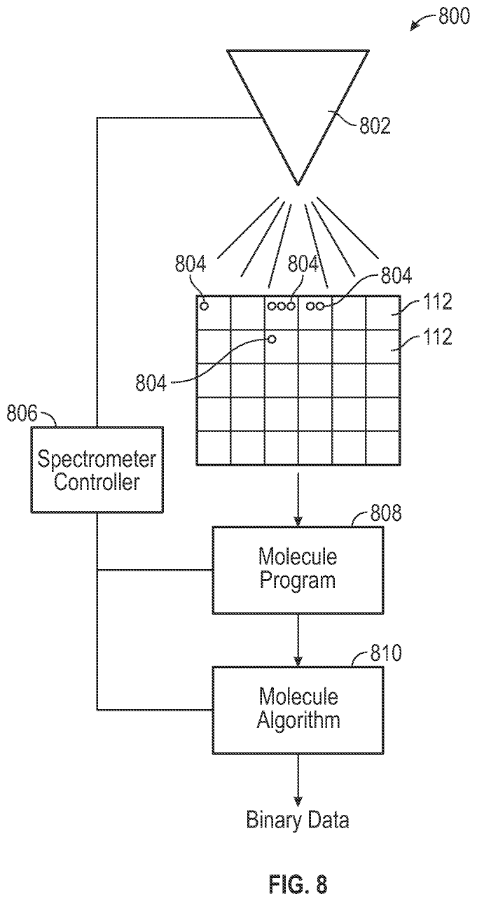

Data stored in the form of molecules on a data storage medium such as 102 of FIGS. 1 and 5 may be read utilizing any suitable molecule identification technique. One example technique for identifying stored molecules is shown in FIG. 8, which employs a spectrometer 800 to identify molecules stored within different grid elements 112 of data storage medium 102. A spectrometer controller 806 may control the different operations of spectrometer 800 in different embodiments. In one embodiment, light from source 802 is directed to grid elements 112 with molecules 804. As light passes through each grid 112 with molecules 704, patterns are generated using a molecule program 808 of spectrometer 800. A molecule algorithm 810 within molecule identifier 706 reads the generated patterns, and matches the patterns against a molecule word library (such as 115 of FIG. 1), and generates data corresponding to the molecules in, for example, a binary format. In some embodiments, a reader (such as 108 of FIG. 1) may identify different combinations of plant-based molecules deposited on a data storage medium (such as 102 of FIG. 1) by ultraviolet spectroscopy, by molecular weight, or by a combination thereof.

The above examples describe storage of single words as molecules, and the retrieval of stored single words by identifying the molecules. In general, data storage systems in accordance with embodiments of the disclosure may be employed for storing any amounts of data. For example, if there are in total 500-1000 grid elements in the data storage medium, by using 22 amino acids in different combinations, in total 500-1000 molecules can be created. In general, molecule generation may be carried out once and may be dependent on a number of grid elements. As indicated in an example provided above, a mixture of three amino acids (R+N+D) may store one byte of information which represents one molecule. A larger combination of amino acids can stretch up to larger data sets (e.g., 2⁶). It should be noted that the grid elements on a data storage medium are labelled/numbered to enable tracking of molecules for storage and retrieval of information.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be reduced. Accordingly, the disclosure and the figures are to be regarded as illustrative rather than restrictive.

One or more embodiments of the disclosure may be referred to herein, individually and/or collectively, by the term “invention” merely for convenience and without intending to limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments employ more features than are expressly recited in each claim. Rather, as the following claims reflect, inventive subject matter may be directed to less than all of the features of any of the disclosed embodiments.

The above-disclosed subject matter is to be considered illustrative, and not restrictive, and the appended claims are intended to cover all such modifications, enhancements, and other embodiments, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A data storage system comprising:
a freeze-tolerant data storage medium, the freeze-tolerant data storage medium having a grid comprising a plurality of grid elements, with each grid element of the plurality of grid elements comprising a storage container;
a molecule generator configured to generate a plurality of different plant-based molecules representing data; and
a molecule depositor, coupled to the molecule generator, configured to deposit different combinations of the plurality of different plant-based molecules into different storage containers of the plurality of storage containers.

2. The data storage system of claim 1 and further comprising a reader configured to identify the different combinations of the plurality of different plant-based molecules deposited into the different storage containers of the plurality of storage containers.

3. The data storage system of claim 2 and wherein the reader is configured to identify the different combinations of the plurality of different plant-based molecules deposited into the different storage containers of the plurality of storage containers by ultra violet spectroscopy, by molecular weight, or by a combination thereof.

4. The data storage system of claim 1 and wherein the freeze-tolerant data storage medium comprises a glass plate, with at least one surface of the glass plate being coated with an antifreeze layer.

5. The data storage system of claim 4 and wherein the antifreeze layer comprises an antifreeze protein.

6. The data storage system of claim 1 and further comprising a molecule library that comprises a mapping of the plurality of different plant-based molecules to corresponding alphanumeric characters represented in binary.

7. The data storage system of claim 6 and further comprising a controller configured to receive binary data, and configured to determine plant-based molecules of the plurality of different plant-based molecules corresponding to the received binary data from the molecule library.

8. The data storage system of claim 7 and wherein the controller is further configured to communicate the determined plant-based molecules to the molecule generator for generation by a chemical reaction.

9. A method comprising:
providing a substrate;
coating at least one surface of the substrate with an antifreeze layer; and
providing a plurality of storage containers on the substrate, the plurality of storage containers configured to store different combinations of a plurality of different plant-based molecules representing data.

10. The method of claim 9 and wherein the substrate comprises a glass plate.

11. The method of claim 9 and wherein the antifreeze layer comprises an antifreeze protein.

12. The method of claim 9 and further comprising arranging the plurality of storage containers within a grid on the substrate, with each grid element comprising a different storage container of the plurality of storage containers.

13. A data storage medium comprising:
a substrate;
an antifreeze layer coated on at least one surface of the substrate; and
a plurality of storage containers on the substrate, the plurality of storage containers configured to store different combinations of a plurality of different plant-based molecules representing data.

14. The data storage medium of claim 13 and wherein the substrate comprises a glass plate.

15. The data storage medium of claim 13 and wherein the antifreeze layer comprises an antifreeze protein.

16. The data storage medium of claim 13 and the plurality of storage containers is arranged within a grid on the substrate, with each grid element comprising a different storage container of the plurality of storage containers.

17. A data storage system comprising the data storage medium of claim 13, the data storage system further comprising:
a molecule generator configured to generate the plurality of different plant-based molecules representing data; and
a molecule depositor, coupled to the molecule generator, configured to deposit different combinations of the plurality of different plant-based molecules into different storage containers of the plurality of storage containers.

18. The data storage system of claim 17 and further comprising a reader configured to identify the different combinations of the plurality of different plant-based molecules deposited into the different storage containers of the plurality of storage containers.

19. The data storage system of claim 18 and wherein the reader is configured to identify the different combinations of the plurality of different plant-based molecules deposited into the different storage containers of the plurality of storage containers by ultraviolet spectroscopy, by molecular weight, or by a combination thereof.

20. The data storage system of claim 17 and further comprising a molecule library that comprises a mapping of the plurality of different plant-based molecules to corresponding alphanumeric characters represented in binary.
